(19) United States
(12) Patent Application Publication
(10) Pub. No.: US 2004/0156919 A1
(43) Pub. Date: Aug. 12, 2004

(54) Drug delivery systems and treatments using them

(76) Inventor: Hoon-Bum Lee, Seoul (KR)

Correspondence Address: GREENBLUM & BERNSTEIN, P.L.C., 1950 ROLAND CLARKE PLACE, RESTON, VA, 20191, US

(21) Appl. No.: 10/483,418

(22) PCT Filed: Jul. 18, 2002
(86) PCT No.: PCT/KR02/01352

(30) Foreign Application Priority Data
Jul. 18, 2001 (KR) 2001/43013

(51) Int. Cl. A61K 33/26

(52) U.S. Cl. 424/646

(57) ABSTRACT

The present invention relates to a drug delivery system of concentrating drugs on a lesion and a method of treating diseases using them. More particular, the present invention provides the delivery system of injecting magnetized drugs into blood vessels of a human body and then applying a magnetic field around the lesion to concentrate the drugs on or around the lesion. Since drugs having been gathered around the lesion occlude micro vessels or are accumulated thereon to be slowly and continuously released, the long-therm therapy can be performed by one-time administration of drugs. Such therapy effect is useful especially for the therapy of cancer cells by anticancer drugs.

### TECHNICAL FIELD

The present invention relates to a drug delivery system of concentrating drugs on a lesion and a method of treating diseases using them. More particular, the present invention provides the delivery system of injecting magnetized drugs into blood vessels of a human body and then applying a magnetic field around the lesion to concentrate the drugs on or around the lesion so that the concentrated drugs would be contiuously released.

### BACKGROUND ART

The human body is an organism in which the blood is circulated to provide tissues and organs with oxygen and nutriment and instead to remove waste materials therefrom. When a specific region in the human body is attacked, leukocytes and lymphocytes gather around the lesion to kill germs. One of so far achievements in science is the invention of various drugs capable of curing diseases to prolong a life, and many new drugs continue to be developed. Methods of efficiently administering such drugs to the human have also been developed, they are diverse depending upon the kinds of diseases and the properties of drugs. Some of such examples are described as below.

U.S. Pat. No. 5,372,579 discloses an electrophoretic/electro-osmotic transdermal drug delivery system for passing drugs through the skin membrane of a patient. In this patent, the system includes a current oscillator that applies periodic electrical variations to the system in order to trigger rhythmical variations of the potential and resistance of the skin membrane so as to cause oscillatory electroWO osmotic streaming of the liquid with the therapeutic compound across the skin membrane in synchronization with the oscillator to the systemic blood of the patient in response to the rhythmical variations.

U.S. Pat. No. 5,403,595 discloses a method of treating a patient for nicotine dependence, which comprises administrating subcutaneously, intramuscularly or by implantation at least one drug delivery system to the patient. In this patent, the drug delivery system comprises at least one microparticle having a composition of lobeline in a biodegradable polymer which releases an effective amount of lobeline to diminish the patient's desire for nicotine for a period of time having a duration of at least one day.

U.S. Pat. No. 6,100,338 discloses particulate carriers useful as drug carriers in a drug delivery system and pharmaceutical compositions making use of such carrier. In this patent, as the particulate carrier, is used the graft copolymer whose graft chain is poly N-alkylacrylamide, poly N-alkylmethacrylamide chain, etc., and it has been reported that the pharmaceutical composition that makes use of the particulate carriers exhibits an excellent peroral absorption enhancement effect of the drug incorporated in the composition.

WO 99/29302 discloses a drug delivery system with two-step targeting, which comprises a combination of (a) a lipid carrier provided with cell targeting agent(s) to target the drug delivery system to specific cells or tissues, and (b) a drug enclosed in the lipid carrier and provided with a DNA targeting agent to target the drug to the nuclei of specific target cells.

Despite these various methods, cancer therapies using anticancer drugs have not been entirely accomplished. This is caused by the characteristics of cancers themselves. Caner cells are mostly identical with normal cells other than the aspect that the rapid cell division and multiplication occur in the cancer cells. As such, most of anticancer drugs is configured to inhibit the synthesis of nucleic acid, being the base of gene in a cell, or bind directly to the nucleic acid to damage the function thereof. However, these drugs do not only affect cancer cells but also normal cells, especially, tissue cells under the active cell division, so that adverse effects are caused, such as bone marrow depression, gastrointestinal mucosa injury, alopecia, etc. Accordingly, the most serious problem in use of general anticancer drugs is that these drugs do not have the specificity about cancer cells but affect all normal cells being under the rapid cell division or multiplication, thereby, damaging the very active cells (marrow cell, gastrointestinal epithelial cell, hair follicles cell, etc.) to cause the bone marrow depression, disturbance gastrointestinal, alopecia, etc. in most of patients. The effects of anticancer drugs on the normal cells and cancer cells are different in aspect of quantity rather than quality. That is, the cancer cells react to the anticancer drug more sensitively than the normal cells to be destroyed in a large number thereof, whereas the normal cells have the high regeneration rate, whereby the therapy effect can be obtained by the anticancer drug.

Meanwhile, referring to FIG. 1 which shows the cell cycle of a normal cell and cancer cell, the gap terminology divides the cell cycle into phases M, G₁, S, and G₂. M (Mitosis) is the period of cell division. G₁ is the period of normal cell metabolism but without replicative DNA systhesis; cells that stay in G₁ for long periods are often referred to as being in the G₀ phase. The S, or DNA synthetic, phase is the period of doubling of the DNA content; it is followed by the G₂, or tetraploid, phase which precedes cell division. Normal and cancer cells have similar cycle times, in general: M, 0.5 to 1 h; G₁, 2 h to infinity; S, 6 to 24 h; G₂, 2 to 8 h. In other words, the times required for the S, G₂ and M phases are generally constant but the time required for the G₁ phase are very different depending on the type of cells, so that the whole time required for the cell division can be said to be determined by the G₁ phase.

Most of anticancer drugs act as inhibiting the replication, transcription, and/or translation processes of DNA, and are divided into six types by the operative mechanism and chemical structure: alkylating agents, antimetabolites, antitumor antibiotics, plant alkaloids, hormones, and miscellaneous agents. In administration of a anticancer drug, the amount of a drug which substantially affect cancer tissues (C×T) is determined by the concentration of drug (C) and the working time (T), and they depend upon pharmacodynamic factors in human subjects.

The most important pharmacodynamic factor is the amount of a drug, but administration way, absorption, transportation and distribution in the body of a drug, metabolism, excretion, and interaction of drugs are also important factors. Furthermore, what must be considered in selection of drugs is in what ways these drugs should be combined in order to prevent the drug resistance by mutagenesis, and in what concentration drugs should be administered.

However, the G₁ phase may continue for a very long time and, in this case, any drug cannot specifically act on the cancer cells of a specific organ. Thus, the cancer cells have not been entirely destroyed, and some survived cancer cells could metastasize to other organs or tissues to cause recurrence of the cancer. For example, approximately one billion cancer cells make up a lump with the diameter of one centimeter. Even where a part of them metastasizes to other organs or tissues, the cancel would recur. When anticancer drugs are administered for a long time in order to prevent the recurrence of cancel, many adverse effects as mentioned earlier would be caused.

### SUMMARY OF INVENTION

The objects of the present invention are to solve the problems described above for once and all.

An object of the present invention is to provide a drug delivery system capable of improving the therapy effect by concentrating drugs such as general anticancer drugs on a lesion (e.g., carcinogenesis region).

A further object of the present invention is to provide a drug delivery system of necrotizing cells of a lesion by occluding micro vessels toward the lesion with drugs.

Another object of the present invention is to provide a drug delivery system of continuously releasing drugs concentrated (gathered) around a lesion for a long time without follow-up measures to improve the therapy effect.

A still another object of the present invention is to provide a drug delivery system capable of affecting cancer cells, being now not in the mitosis phase, when they start to enter the this phase in the future, by continuously releasing drugs concentrated around a cancer region.

In order to accomplish these objects, a drug delivery system according to the present invention comprises, a drug means being of a particle form and being provided with ferromagnetism or ferrimagnetism (“particle-typed magnetic drug means”), and an external magnetic field-generating (“MFG”) means of generating the magnetic field around a lesion in injection of the particle-typed magnetic drug means into a blood vessel to concentrate the particle-typed magnetic drug means on the lesion.

According the drug delivery system of the present invention, the particle-typed drug means with ferromagnetism or ferrimagnetism is injected into a feeding vessel of a patient and then moves toward the lesion by the magnetic field generated from the MFG means which has been positioned around the lesion. It allows a magnetic drug means, although it has been injected in a small amount, to be highly concentrated around the lesion to improve the therapy effect.

The types of drugs useful for the present invention are not particularly limited, but drugs such as anticancer drugs without the specificity about a lesion (caner cells), i.e., drugs also making adverse effects on normal cells, are especially useful. Providing the particle-typed drugs with ferromagnetism or ferrimagnetism can be performed in various ways. For example, for general particle-typed drugs composed of an active component and carrier, (a) a magnetic material with ferromagnetism or ferrimagnetism is mixed in a carrier or used as a carrier, (b) a magnetic material is coated at least on a part of the outer surface of a drug, or (c) a magnetic metal ion with ferromagnetism or ferrimagnetism is directly bound to the active component by chemical reaction. In the way (b), the coating of the magnetic material can be preferably performed by coating the outer surface of a drug with a biodegradable material in which the magnetic material is mixed. One desirable example of the biodegradable materials is lipid. As known in the art to which the present invention pertains, a drug coated with the lipid is not harmful for a human subject, and it takes much times until the lipid is decomposed entirely, which allows the drug not to lose the magnetism until the drug reaches a lesion. In the way (c), the chemical bond of the magnetic metal ion to the active component can be accomplished by binding materials, having the magnetism even in ion form, through chemical reaction in the synthesis of the active component. Some materials have stronger magnetism in ion form. However, such chemical bond of the metal ion should not diminish the efficacy of drug. Some of existing drugs are made of metal complexes of transition metal ions. Where the metal complex has ferromagnetism or ferrimagnetism, these drugs can be used as the magnetic drug means of the present invention. Micro blood vessels in a human subject have approximately the diameter of 3 to 7 μm. Therefore, the maximum diameter of the drug particles should be the same as or below 3 μm being the minimum diameter of micro vessels, and preferably, is 0.1 to 2 μm.

The magnetic properties of materials stem from the spin arrange of electrons in 3d or 4f orbital and can be divided into (i) paramagnetism wherein the directions of spins are random, (ii) ferromagnetism wherein the directions of spins are toward one side, (iii) ferrimagnetism wherein the directions of spins are alternatively toward one side and the opponent side but the direction toward one side is predominant, and (iv) antiferromagnetism wherein the directions of spins are alternatively toward one side and the opponent side. Materials with the ferromagnetism or ferrimagnetism can be attracted to the MFG means such as a magnet, whereas materials with the paramagnetism or antiferromagnetism cannot be attracted. Accordingly, the material which is mixed in the particle-typed drug or coated thereon should have the magnetic property of ferromagnetism or ferrimagnetism as well as the biocompatible property (i.e., even where it remains in a human subject, it should be not harmful). Such a representative material is iron.

The magnetic field-generating (MFG) means works as generating the magnetic field to attract the magnetic drug means around a lesion, and the representative ones thereof are a permanent magnet and an electromagnet.

The types of magnets are various, such as alnico magnet, ferrite magnet, rare earths magnet, etc. For the alnico magnet, the residual induction (Br) is high but the coercive force (bHc) is low, so that the energy product is small. A magnet with a low coercive force is apt to be demagnetized, and thus it is required to be magnetized in the phase that the magnetic circuit is completed. On the other hand, the ferrite magnet, and rare earths magnet such as Nd-based magnet, Sm-based magnet, Ce-based magnet have a high coercive force, and thus they can be magnetized as a magnet single body.

The electromagnet is a magnet which becomes magnetized upon application of an electric current and becomes demagnetized upon interruption of an electric current, and is generally made in a configuration that a conductive bar such as soft iron bar is inserted in the interior of a round coil. A magnetic field is generated at the surrounding of a coil through which an electric current passes, and the direction of the magnetic field is a clockwise direction against the direction of the electric current. Therefore, the electromagnet can generate the magnetic field only during application of the electric current.

The MFG means can be made in various configurations. The simplest configuration comprises a bar-type body and a permanent magnet or electromagnet installed at the end of the bar-type body. Of course, another additional configurations being able to concentrate the magnetic field are possible. The magnetic field generated from the external MFG means needs not be restricted to only a lesion but may reach tissues and/or organs around the lesion. That is because, for example, where cancer cells have also been spread on the vicinity of a specific region (lesion) detected by an examination, anticancer drugs can reach that vicinity, and furthermore, as mentioned below, the anticancer drugs gathered around the lesion are continuously released to reach the lesion by the special effect of the present invention.

Cancer cells multiply more rapidly than normal cells, which requires sufficient feeding of oxygen, nutriment, etc. For such sufficient feeding, new micro vessels are concentratedly generated (“self-multiply”) around a cancer cell region by new vessel-inducing materials secreted from cancer cells, which is so called “neovascularization.” Meanwhile, according to the defense mechanism of a human body, another new micro vessels are also generated (“self-defend”) to send leukocytes and lymphocytes to the cancer cells. By the drug delivery system according to the present invention, the magnetic drug means goes to the cancer cells through these new micro vessels.

One of important features in the present invention is for the particle-typed drugs to necrotize the cancer cells by occluding these new micro vessels toward the cancer cells. As mentioned earlier, because the maximum diameter of the particle-typed drugs is the same as or smaller than the minimum diameter of the micro vessels, one particle-typed drug cannot entirely occlude the micro vessel; however, where a plurality of particle-typed drugs are accumulated on a specific region of the micro vessel, the blood flow can become blocked. Such accumulation can be accomplished by the particle-typed drugs themselves or by agglutination of drugs and blood components such as erythrocyte, thrombocyte, etc. Resultantly, the blood feeding to the cancer cells is interrupted so that the cancer cells, being under the active cell division and multiplication, become necrotized.

Another important feature of the present invention is that magnetic drugs, having been gathered on or around a lesion, are slowly and continuously released, which means that the drug efficacy can be kept for a long time. Such slow and continuous release is useful especially for anticancer drugs. As mentioned earlier, most of anticancer drugs work only on mitotic cancer cells but not on cancer cells generally being under G₁ or G₂ phase. Therefore, in order to entirely destroy caner cells of which the entrance time into the mitotic phase cannot be anticipated, anticancer drugs need to be continuously administered, which also makes an adverse effect on normal cells. In particular, it cannot be basically prevented that cancer cells being under a quiescent phase may metastasize to another region. Accordingly, the sustained release, being a special effect according to the drug delivery system of the present invention, can be said to be very useful for the cancer therapy by anticancer drugs. In another embodiment, where the magnetic drug means itself is made as the sustained release formulation, the sustained release effect is further improved by the drug delivery system of the present invention.

The present invention also provides a method for treatment of diseases by concentrating drugs on a lesion using the drug delivery system. The treatment method comprises,

(1) a particle-typed drug means with ferromagnetism or ferrimagnetism (“magnetic drug means”) is injected into a blood vessel in which the blood flow is toward a lesion;

(2) the magnetic field is generated on or around the lesion by an external magnetic field-generating (MFG) means to concentrate the magnetic drug means on or around the lesion; and,

(3) the magnetic drug means, having been concentrated on or around the lesion, is directly sent to the lesion, or is accumulated on a partial region of a micro blood vessel to occlude the vessel or to be slowly and continuously released to the lesion, so that the lesion is cured.

Generating the magnetic field on the lesion can be performed by various ways;

for example, a way of positioning the external MFG means outside a human body to generate the magnetic field directly on the lesion, a way of inserting the external MFG means into a human body by an endoscope to generate the magnetic field directly on the lesion, etc. The latter is especially useful for the case where cancer cells were found on organs, such as stomach, lung, rectum, etc., leading to the exterior of a human body.

As shown below, the description refers to the drawing in order to describe the present invention more in detail, thereby, the scope of the invention is however not to be interpreted as a limitation of the invention.

### DETAILED DESCRIPTION OF PREFERRED EMBODIMENTS

FIG. 2 shows one embodiment according to the drug delivery system of the present invention. Particle-typed anticancer drugs with ferromagnetism are suspended or emulsified in a saline solution to prepare an injection 110. The injection 110 is filled in a syringe 100 and then injected in a feeding vessel 200 through the syringe 100. The injecting position is selected, if possible, in the vicinity of a cancer region 300 and on a vessel in which the blood flow is toward the cancer region 300. Meanwhile, a MFG device 400 with a magnet 410 at the end thereof is positioned outside the cancer region 300. The figure shows the configuration that a body skin 500 is pressed by the magnet 410 in order to position the magnet 410 near to the cancer region 300 but, if necessary, the body skin 500 is incised and then the magnet 410 is inserted through the body skin 500, whereby the magnet 410 can be further approached to the cancer region 300. In this case, it is necessary that the feeding vessel 200 should not be positioned on the path through which the magnet 410 is inserted. That is because, where the feeding vessel 200 is positioned on that path, the magnetic drugs may gather around the magnet 410 rather than the cancer region 300.

Many micro vessels 600 from feeding vessels 200 and their branch vessels 210 are newly made around the cancer region 300. The injection 110, having been injected into the feeding vessel 200 through the syringe 100, enters the cancer region 300 through the micro vessel 600. The driving force of delivering the injection 110 to the cancer region 300 is the attraction of the magnetic drugs by the magnetic field of the magnet 410, and the absorption of the blood for the feeding of nutriments by the cancer region 300.

FIG. 3 shows the phenomenon of delivering magnetic drugs to a cancer region through micro vessels.

Most of magnetic drugs, flowing in a feeding vessel 200, move toward a cancer region 300 through micro vessels 600 which are connected to the cancer region 300.

The micro vessels 600 have generally smaller diameters than the feeding vessel 200, and the partial region of a micro vessel 600 has a further smaller diameter. Accordingly, the magnetic drugs 700, flowing into the cancer region 300 through the micro vessel 600, are apt to accumulate on a narrow part, which can be called as “bottle neck phenomenon.” As seen in FIG. 3, accumulation of the magnetic drugs 700 generally occurs at the entrance 610 and a narrower part 620 of micro vessel 600.

As the result of accumulation, new supply sources of magnetic anticancer drugs 700 may be made (as in 610, 620), or the micro vessel 600 may be entirely occluded (as in 630). These phenomena can be sustained even after the magnetic field around the cancer region 300 is removed. Accordingly, in the former (as in 610, 620), the accumulated magnetic anticancer drugs 700 are slowly and continuously released to move toward the cancer region 300. Where the anticancer drugs 700 themselves have been formulated as a sustained-release drug, the above continuous release effect is further irnproved, so that the long-tern therapy can be performed by one-time administration of the anticancer drug. In the latter (as in 630), since the blood for feeding oxygen and nutriment to the cancer region 300 is entirely blocked, the cancer region 300 with a rapid multiplication and growth becomes necrotized.

The present invention being thus described, it will be obvious that the same may be varied in many ways. Such variations are not to be regarded as a departure from the spirit and scope of the invention and all such modifications would be obvious to one skilled in the art.

### BRIEF DESCRIPTION OF THE DRAWINGS

FIG. 1 is a view of the cell cycle of a normal and cancer cells, which shows the procedure of the cell division and growth.

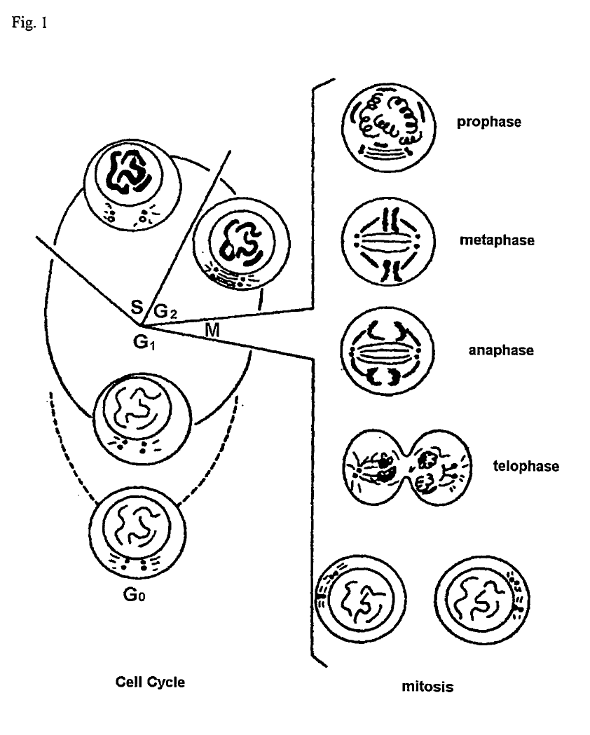

FIG. 2 is a perspective view of a drug delivery system according to an embodiment of the present invention.

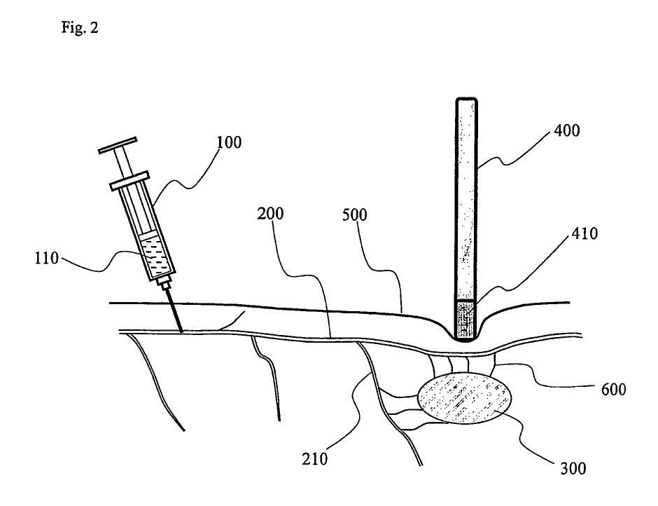

FIG. 3 is a perspective view of particle-typed drugs gathering around a cancer region, in which many drugs are accumulated on micro vessels toward the cancer region as the effect of the present invention.

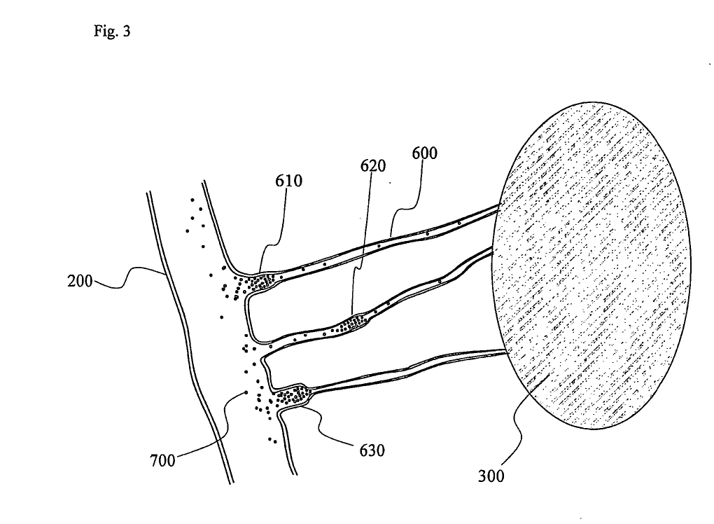

Designation of the Reference Numbers

100: syringe 200: feeding vessel

300: cancer region 400: magnetic field-generating device

500: body skin 600: micro vessel

700: particle-typed magnetic drug

### INDUSTRIAL APPLICABILITY

The drug delivery system of the present invention provides the high density of drugs around a lesion with a small amount of drugs administered, and by administration of a small amount of drug, the adverse effects of drug and the relevant complication can be remarkably diminished or prevented. Moreover, since drugs having been gathered around the lesion occlude micro vessels or are accumulated thereon to be slowly and continuously released, the long-term therapy can be performed by one-time administration of drugs. Such therapy effect is useful especially for the therapy of cancer cells by anticancer drugs.

What is claimed is:

1. A drug delivery system comprising a drug means being of a particle form and being provided with ferromagnetism or ferrimagnetism (“particle-typed magnetic drug means”), and an external magnetic field-generating (“MFG”) means of generating the magnetic field around a lesion in injection of the particle-typed magnetic drug means into a blood vessel to concentrate the particle-typed magnetic drug means on the lesion.

2. The drug delivery system according to claim 1, wherein the drug means is a anticancer drug.

3. The drug delivery system according to claim 1, wherein the particle-typed drug means is provided with ferromagnetism or ferrimagnetism by one of the below ways: (a) a magnetic material with ferromagnetism or ferrimagnetism is mixed in a carrier or used as a carrier, (b) a magnetic material is coated at least on a part of the outer surface of a drug, and (c) a magnetic metal ion with ferromagnetism or ferrimagnetism is directly bound to the active component by chemical reaction.

4. The drug delivery system according to claim 3, wherein the coating of the magnetic material in the way (b) is performed by coating the outer surface of a drug with a biodegradable material in which the magnetic material is mixed.

5. The drug delivery system according to claim 4, wherein the biodegradable material is lipid.

6. The drug delivery system according to claim 1, wherein the maximum diameter of the drug particle is in the range of 0.1 to 2 μm.

7. The drug delivery system according to claim 1, wherein the MFG means is a permanent magnet or electromagnet.

8. The drug delivery system according to claim 1, wherein the magnetic drug means is formulated as a sustained-release drug.

9. A method for treatment of diseases by concentrating drugs on a lesion comprising,
(1) a particle-typed drug means with ferromagnetism or ferrimagnetism (“magnetic drug means”) is injected into a blood vessel in which the blood flow is toward a lesion;
(2) the magnetic field is generated on or around the lesion by an external magnetic field-generating (MFG) means to concentrate the magnetic drug means on or around the lesion; and,
(3) the magnetic drug means, having been concentrated on or around the lesion, is directly sent to the lesion, or is accumulated on a partial region of a micro blood vessel to occlude the vessel or to be slowly and continuously released to the lesion, so that the lesion is cured.

10. The method according to claim 9, wherein generating the magnetic field on the lesion is performed by a way of positioning the external MFG means outside a human body to generate the magnetic field directly on the lesion, or a way of inserting the external MFG means into a human body by an endoscope to generate the magnetic field directly on the lesion.
